(19) United States
(12) Patent Application Publication
(10) Pub. No.: US 2024/0209419 A1
(43) Pub. Date: Jun. 27, 2024

(54) Systems and Methods for Capture and Enrichment of Clustered Beads on Flow Cell Substrates

(71) Applicant: Illumina, Inc., San Diego, CA (US)

(72) Inventors: Naneki McCallum, San Diego, CA (US); Fei Shen, San Diego, CA (US); Xiaoyu Ma, San Diego, CA (US); Weixian Xi, San Diego, CA (US); Olivia Ghazinejad, San Diego, CA (US); Jeffrey Brodin, San Diego, CA (US); Jeff Fisher, San Diego, CA (US)

(73) Assignee: Illumina, Inc., San Diego, CA (US)

(21) Appl. No.: 18/539,988

(22) Filed: Dec. 14, 2023

Related U.S. Application Data
(60) Provisional application No. 63/432,537, filed on Dec. 14, 2022.

(51) Int. Cl. C12Q 1/6818; C12Q 1/6874

(52) U.S. Cl. CPC C12Q 1/6818; C12Q 1/6874; C12Q 1/6851

(57) ABSTRACT

Methods for on-flow cell selective capture and enrichment of clustered beads, general capture strategies on bead mobility on flow cell surfaces, sorting clustered and unclustered beads, and flow cell reusability for bead immobilization onto flow cells.

### CROSS-REFERENCE TO RELATED APPLICATIONS

This application claims priority to U.S. provisional patent application No. 63/432,537, filed Dec. 14, 2022, the entire disclosure of which is hereby incorporated by reference herein in its entirety.

### BACKGROUND

Cluster generation is a key processing step in the sequencing of DNA and other nucleic acid materials using Next Generation Sequencing (NGS) technologies. Sequencing methodologies for DNA targets on NGS platforms commonly deploy DNA libraries in which DNA (e.g., gDNA or cDNA) is processed into fragments and ligated with technology-specific adaptors. NGS workflow on, e.g., for DNA on Illumina NextSeq® series platforms, involves loading a DNA library onto a flow cell and hybridizing individual DNA fragments to adapter-specific complimentary oligonucleotides (oligos) covalently bound to the flow cell surface (planar surface or nano-well surface); clustering the individual fragments into thousands of identical DNA template strands through bridge or non-bridge amplification techniques; and, finally, sequencing, in which copy strands are simultaneously synthesized and sequenced on the DNA templates (Sequence-by-Synthesis (SBS)) using a reversible terminator-based process that detects signals emitted from fluorophore-stained single bases as they are added round by round to the copy strands. Because the multiple template strands of each cluster have the same sequence, base pairs incorporated into the corresponding copy strands in each round will be the same, and thus the signal generated from each round will be enhanced proportional to the number of copies of the template strand in the cluster.

Flow cells commonly support multiple chemistries and analyses across the workflow from the same or adjacent functionalized space, making it difficult to perform any one function under a set of conditions optimized to that particular function. Moreover, chemistries supported on the flow cell for one function may interfere with or degrade flow cell chemistries needed to support other functions. For example, numerous cycles of use and the associated reactions that take place to functionalize the flow cell surface for cluster generation can degrade the capacity of the flow cell to maintain the surface chemistry necessary for various analyses, including retention of signal intensity necessary for base calling, and in some cases flow cells can be simply considered a consumable. As in most industries, fewer consumables in a process are desirable.

### SUMMARY

Various embodiments described herein include methods and compositions for flow-cell based sequencing of clonal populations of a nucleic acid library clustered on an array of bead substrates; orthogonal reagents and complimentary chemistry for functionalization of a flow cell surface for selective capture, in situ enrichment, imaging, and traceless release of clustered beads in a sequencing cycle; kits comprising flow cells, beads and reagents for use with the methods described herein; and systems and devices for performing such methods. Embodiments described herein provide nanoscale to microscale beads as high surface area substrates for in-solution cluster generation, in which flow cell chemistry and architecture is decoupled from the clustering process. Functionalized flow cells in accordance with certain embodiments retain signal intensity levels over numerous cycles of analysis.

Certain embodiments provide reagents for efficient capture and traceless release of clustered beads during a sequencing cycle. The chemistry used to immobilize clustered beads onto a flow cell surface is preferably orthogonal to the hybridization-based chemistry used to functionalize the bead substrate such that clustered beads may be selectively cleaved under conditions that conserve flow cell surface chemistry for repeated reuse. For example, according to certain embodiments, immobilization of clustered beads onto a flow cell surface is affected through conjugation of individual amplicons with a capture agent and complementary binding partners coated on the flow cell surface. For some embodiments, biotin and avidin moieties serve as respective binding agents and partners. In other, preferred embodiments, biotin and streptavidin moieties serve as respective binding agents and partners. In one example, amplicons are selectively, site-specifically chemically modified to incorporate biotin moieties during the clustering process through inclusion of biotinylated adaptor primers in a solution comprising primer mix having a desired ratio of adaptor primer to biotinylated analog. After immobilization and sequencing, cleaving reagents for traceless release of the biotin ligand from streptavidin conjugates may be introduced to cleave immobilized beads from the flow cell surface after the completion of a cycle of analysis under conditions that conserve functionality of the avidin coating for a following cycle.

In one example, a method of performing a cycle of sequencing analysis is provided that includes clustering, in a solution or suspension, a clonal amplicon population from each of a plurality of nucleic acid targets seeded to an array of bead substrates, wherein a desired portion of each clonal amplicon population is conjugated with a biotin moiety, and wherein clustering results in a pool of clustered, biotinylated bead substrate. The method further comprises providing a flow cell substrate functionalized with a coating of streptavidin moieties on at least a portion of the substrate, wherein each avidin moiety has one or more ligand domains characterized by a high affinity for binding biotin, loading the pool of clustered, biotinylated bead substrates and immobilizing at least a portion of the biotinylated bead substrates onto the flow cell substrate via interactions between biotin moiety conjugates of the biotinylated beads substrates and the streptavidin ligand domains of the coating of avidin moieties. For instance, a respective biotin moiety may interact with a respective ligand domain to form an orthogonal complex between an amplicon conjugated with the respective biotin moiety and functionalized substrate of the flow cell. The method may further include performing sequence analysis on the immobilized bead substrates. The method may also include introducing a cleaving reagent to remove the immobilized substrates at the end of an analysis cycle, wherein the cleaving reagent dissociates the orthogonal complex under conditions that substantially conserve functionality of the coating of streptavidin moieties on the flow cell surface for multi-cycle use. In certain embodiments, biotin-streptavidin interaction may be further promoted through conjugation of one or both of the biotin and streptavidin moieties with clickable groups such as azides or linear or cyclic alkynes.

Various embodiments described herein also include methods for the preparation and selective capture of clustered beads having a high degree of both monoclonality and fragment density (i.e., high occupancy). A number of amplification techniques are contemplated for use in connection with the embodiments described herein, including bridge amplification, rolling-circle amplification, emulsion PCR, and exclusion amplification (ExAmp).

Of the available techniques, ExAmp cluster generation is particularly appropriate for optimizing monoclonality of clustered beads. Preparation of a monoclonal cluster on a bead substrate ideally requires 1:1 seeding ratio to ensure the cluster results from amplification of only a single fragment. In reality, however, a 1:1 seeding ratio is difficult to achieve because the variance of the number of seeded fragments per bead is stochastic and, thus, seeding events tend to occur along a Poisson distribution. ExAmp chemistry carries out seeding and cluster amplification steps simultaneously such that amplification of a first seeding event on a given bead occurs nearly instantaneously and the amplification rate far exceeds the reaction rate for seeding. In that manner, the ExAmp amplification of a fragment seeded to a bead excludes (or limits) further seeding by other molecules, thus reducing the occurrence of undesirable polyclonal clustering. Low density seeding may also be performed in conjunction with ExAmp or other amplification techniques in accordance with embodiments disclosed herein to further optimize monoclonality of the clustered bead pool. Low density (sub-Poisson) seeding effectively narrows the probability distribution of stochastic variance and skews the distribution mean toward a 1:1 seeding ratio, which results in increased populations of monoclonal beads in the cluster pool.

In one example, a method (e.g., for amplifying nucleic acid libraries) is provided that includes mixing a first reagent mixture with an amount of double stranded target nucleic acids within a reservoir to define a first solution, wherein the 5′ and 3′ ends of target nucleic acids are ligated with respective first and second adapters, and wherein the first reagent mixture comprises nucleoside triphosphates (NTPs), one or more replication enzymes, and adapter primers, wherein the adaptor primers include a primer mix of second adaptor primers and biotinylated analogs in a desired ratio. The method also includes mixing the first solution with a second solution (or suspension) comprising primer grafted beads, wherein each primer is configured to hybridize to the first adapter of the target nucleic acids. Clustering is performed to produce a pool of clustered beads, wherein target nucleic acids are seeded onto primer grafted beads and amplified to form clonal populations, wherein the rate of amplification rate preferably exceeds the rate at which target nucleic acids are seeded. Each clonal population will comprise amplicons synthesized with either a second adaptor primer or a biotinylated analog, wherein the desired ratio of biotinylated to non-biotinylated amplicons corresponds to the ratio of the primer mix. The method may include one or more additional steps of adding fresh quantities of the NTPs and the one or more replication enzymes to the second solution to increase the number of amplicons in the clonal population on each clustered bead.

While ExAmp and low-density seeding techniques—used alternatively or complimentary—are effective in generating cluster pools having a high degree of monoclonality, an excess of unclustered or poorly clustered beads tend to populate the pool due, in large part, to the exigencies of random diffusion of reagents to bead substrates in solution or suspension. In these instances, a flow cell substrate in accordance with various embodiments described herein may have a surface chemistry adapted to selectively capture well-clustered beads with high affinity relative to unclustered or poorly clustered beads, such that unclustered and poorly clustered beads can be selectively and efficiently removed via, e.g., a waste channel. In certain embodiments, a reaction mixture comprising a capture agent adapted to conjugate an amount of amplicons in a population and a flow cell substrate having a complementary chemistry of a given density are refined in a manner that enables well-clustered beads to selectively bind the flow cell substrate with optimal affinity; whereas unclustered beads cannot bind, and the poorly clustered beads have a low binding affinity, and are thus less likely to efficiently bind, due to the relative scarcity of conjugated capture agents, allowing for selective removal through a simple wash step.

In one such embodiment, the capture moiety is or comprises a biotin moiety and the complementary chemistry on the cell flow substrate comprises a monolayer of avidin moieties. In one example, a method (e.g., for amplifying nucleic acid libraries) is provided that includes mixing a first reagent mixture with an amount of double stranded target nucleic acids within a reservoir to define a first solution, wherein the 5′ and 3′ ends of target nucleic acids are ligated with respective first and second adapters, and wherein the first reagent mixture comprises nucleoside triphosphates (NTPs), one or more replication enzymes, and adapter primers, wherein the adaptor primers include a primer mix of second adaptor primers and biotinylated analogs in a desired ratio. The method also includes mixing the first solution with a second solution (or suspension) comprising primer grafted beads, wherein each primer is configured to hybridize to the first adapter of the target nucleic acids. Clustering is performed to produce a pool of clustered beads, wherein target nucleic acids are seeded onto primer grafted beads and amplified to form clonal populations, wherein the rate of amplification preferably exceeds the rate at which target nucleic acids are seeded. Each clonal population will comprise amplicons synthesized with either a second adaptor primer or a biotinylated analog, wherein the desired ratio of biotinylated to non-biotinylated amplicons may be obtained using a corresponding ratio the primer mix. Clustering will yield a bead pool comprising well-clustered beads characterized by high biotinylation, poorly clustered beads characterized by low biotinylation relative to the first constituent group, and unclustered beads lacking biotinylation. After one or more clustering steps are performed, the bead pool is loaded onto a flow cell substrate functionalized with a monolayer of avidin moieties that bind biotin with high affinity such that well-clustered beads of the bead pool are selectively captured on the monolayer, which enables sorting of poorly clustered and unclustered beads from the bead pool.

Target nucleic acid fragments may be cloned into single stranded or double stranded amplicons. Double stranded amplicons in accordance with various embodiments described herein may be functionally cross-linked to withstand de-hybridization conditions and/or mechanical reagent flow needed for on-flow cell sequencing. In one example embodiment, complementary clonal strands are cross-linked via 3-cyanovinylcarbazole nucleoside (CNVK).

The various methods and compositions for particle-based, in-solution cluster generation described herein present numerous advantages. The sequencing workflow supported by the disclosed methods herein conserves flow cell chemistry through multiple cycles of use. In-solution (or in-suspension) cluster generation as provided herein simplifies flow-cell chemistry and thus limits interference, cross contamination, and cross-reactivity among the multiple chemistries of the workflow. The complimentary orthogonal chemistries and clonal motifs for use with the disclosed methods generate stable, particle-bound clonal populations having optimized density and high homogeneity to enable high-fidelity sequencing with improved signal-to-noise ratio and phasing/pre-phasing. The bead substrates for use with the disclosed methods have enhanced extensibility such that disclosed methods can be leveraged for use in large particle analysis or with multiomic applications. And Individual addressable clustering supported by disclosed methods eliminates index hopping and other complications that arise during amplification.

Other features, objects, and advantages will be apparent from the following detailed description, accompanying drawings, and the claims. However, it is to be understood that both the foregoing general description and the following detailed description present embodiments of the subject matter of the present disclosure and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF THE DRAWINGS

The foregoing summary, as well as the following detailed description of disclosed embodiments, will be better understood when read in conjunction with the appended drawings. For purposes of illustration, there are shown in the drawings embodiments which are presently preferred. It should be understood, however, that the disclosure is not limited to the precise arrangements and instrumentalities shown. In the drawings, like reference numerals correspond to similar, though perhaps not identical, components. For the sake of brevity, reference numerals or features having a previously described function may or may not be described in connection with other drawings in which they appear.

FIG. 1 shows, in (a), a schematic representation of an example of direct hybridization of a denatured clustered or other single stranded DNA-containing bead to the flow cell, in (b) a schematic representation of an example of hybridization of beads to the flow cell using a “bridge” oligo which spans the region between the bead and flow cell, and, in (c) biotinylation of the beads for bead capture added through hybridization of a biotinylated oligo.

FIG. 2 shows fluorescence images of flow-cell capture of biotinylated, clustered beads on a streptavidin-coated substrate versus flow-cell capture of non-biotinylated clustered beads using direct and indirection hybridization interactions on a complimentary oligo lawn.

FIG. 3 shows a schematic representation of an example of site-specific incorporation of biotin into clustered beads, whereby P7 primer grafted beads are seeded with the library of choice, and then clustered using non-bridging clustering with a mixture of P5 and biotinylated P5 primers in solution.

FIG. 4 shows, in (a), fluorescence images of flow-cell capture of green labeled biotin-incorporated clustered beads amplified with in-solution P5 primer mixes of 0%, 10%, 20%, and 30% biotinylated P5 primer concentrations versus red-labeled P7 control, in (b) a bar graph of measures of average beads calculated from image data from the fluorescence images in (a), and, in (c) a bar graph of measures of molecules per bead based on qPRC analysis.

FIG. 5 shows a scatter plot of relative hybridization during amplification over time for two separate batches of 10 bp DNA clones—one incorporating biotinylated P5 primer and the other incorporating non-biotinylated P5 primer.

FIG. 6 is a schematic representation of an example method of crosslinking via 3-cyanovinylcarbazole nucleoside (CNVK) incorporation including, in (a), a schematic representation of a CNVK crosslinking reaction on captured bead, in (b), a schematic representation of crosslinked beads and non-crosslinked beads under de-hybridization conditions, in (c), the structure of a CNVK nucleoside, and, in (d), a CNVK photo-crosslinking reaction scheme.

FIG. 7 shows a schematic representation of 3′end click chemistry tailing for modification of dsDNA-coated beads including in (a), a general workflow of 3′ tailing, showing digestion of excess lawn primers followed by TdT treatment with modified NTPs to incorporate biotin and, in (b), a schematic showing general possibilities for modified NTP incorporation by TdT and possible subsequent modifications, including click reaction with a wide variety of small molecules, e.g., ligation and primer extensions.

FIG. 8 shows a schematic representation of a click chemistry workflow for biotinylated bead capture on flow cell coated with PAZAM having surface azides serving as sites for clicking on biotinylated alkynes serving as a scaffold for a streptavidin monolayer.

### DETAILED DESCRIPTION

It is to be understood that terms used herein will take on their ordinary meaning in the relevant art unless specified otherwise. Several terms used herein and the meanings encompassed by those terms are set forth below.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

As used herein, the singular terms “a” and “the” are synonymous and used interchangeably with “one or more” and “at least one,” unless the language and/or context clearly indicates otherwise. Accordingly, for example, reference to “a compound” or “the compound” herein or in the appended claims can refer to a single compound or more than one compound. Additionally, all numerical values, unless otherwise specifically noted, are understood to be modified by the word “about.” The terms comprising, including, containing and various forms of these terms are synonymous with each other and are meant to be equally broad.

The terms first, second, etc. also are not meant to imply a specific orientation or order, but rather are used to distinguish one component from another. It is to be understood that the ranges provided herein include the stated range and any value or sub-range within the stated range, as if such values or subranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

For simplicity and clarity of illustration, elements in the figures are not necessarily to scale, and the same reference numbers in different figures denote the same elements.

Certain terminology is used in the following description for convenience only and is not limiting. The words “right”, “left”, “lower”, and “upper” designate directions in the drawing to which reference is made and are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s). The words “inwardly” and “outwardly” refer direction toward and away from, respectively, the geometric center of the object described and designated parts thereof. The terminology includes the words above specifically mentioned, derivatives thereof and words of similar import.

As used herein, “alkyne” or “alkynyl” refers to a straight or branched hydrocarbon chain containing one or more triple bonds. The alkynyl group may have 2 to 20 carbon atoms.

As used herein, the term “attached” or “attaching” refers to the state of two things being joined, fastened, adhered, connected, or bound to each other, either directly or indirectly. For example, a nucleic acid can be attached to a functionalized polymer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions, and hydrophobic interactions.

As used herein, an “azide” or “azido” functional group refers to —N₃.

As used herein, a “bead” refers to any micro- or nano-bead or particle architecture having a substrate or surface that is capable of binding, immobilizing, adhering or otherwise attaching nucleic acid material.

As used herein, the term “capture agent” refers to a material, chemical, molecule or moiety thereof that is capable of attaching, retaining, or binding to a target molecule (e.g., a target nucleic acid). Example capture agents include, without limitation, a capture nucleic acid that is complementary to at least a portion of a target nucleic acid, a member of a receptor-ligand binding pair (e.g., avidin, streptavidin, biotin, lectin, carbohydrate, nucleic acid binding protein, epitope, antibody, etc.) capable of binding to a target nucleic acid (or linking moiety attached thereto), or a chemical reagent capable of forming a covalent bond with a target nucleic acid (or linking moiety attached thereto).

As used herein, the term “clonal population” refers to a population of nucleic acids that is homogeneous with respect to a particular nucleotide sequence. The homogenous sequence may be at least about 10 nucleotides long, but can be even longer including for example, at least about 50, about 100, about 250, about 500 or about 1000 nucleotides long. A clonal population can be derived from a single target nucleic acid or template nucleic acid. Most, if not all, of the nucleic acids in a clonal population have the same nucleotide sequence. It will be understood that a small number of mutations (e.g., due to amplification artifacts) can occur in a clonal population without departing from clonality.

As used herein, the term “CNVK” (3-cyanovinylcarbazole nucleoside) refers to a useful chemical moiety that may be incorporated within double-stranded oligos to undergo rapid photo cross-linking to the complementary strand using a single wavelength trigger. CNVK as used herein has the general formula:

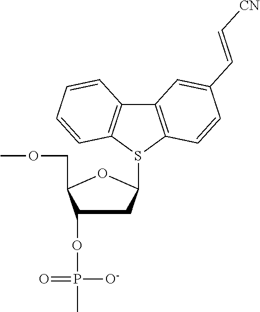

As used herein, the term “diffusion” refers to movement of a molecule through a fluid. The term can include passive diffusion, such as movement of molecules along their concentration gradient. The term can also include active transport, whereby molecules can move along their concentration gradient or against their concentration gradient. Thus, diffusion can include applying energy to move one or more molecules in a desired direction or to a desired location such as a bead surface.

As used herein, the term “double stranded,” when used in reference to a nucleic acid molecule, means that at least substantially all of the nucleotides in the nucleic acid molecule are hydrogen bonded to a complementary nucleotide. A partially double stranded nucleic acid can have at least about 10%, about 25%, about 50%, about 60%, about 70%, about 80%, about 90% or about 95% of its nucleotides hydrogen bonded to a complementary nucleotide.

The term “each,” when used in reference to a collection of items, is intended to identify an individual item in the collection, but does not necessarily refer to every item in the collection. Exceptions can occur if explicit disclosure or context clearly dictates otherwise.

As used herein, the term “flow cell” is intended to mean a vessel having a flow channel that is in fluid communication with at least one unmodified surface or at least one surface modified with a first member of a transition metal complex binding pair. The unmodified or modified surface is capable of attaching surface chemistry that to be used in during a nucleic acid analysis, and is capable of releasing the surface chemistry either electrochemically or upon exposure to visible light. The flow cell also includes an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a patterned or nonpatterned structure and a lid. In other examples, the flow channel may be defined between two patterned or non-patterned structures that are bonded together.

As used herein, the term “NTPs” (nucleoside triphosphates) refers to a nucleotide containing a nitrogenous base bound to a 5-carbon sugar (e.g., ribose or deoxyribose), with three phosphate groups bound to the sugar.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

In some examples, the term “over” may mean that one component or material is positioned directly on another component or material. When one is directly on another, the two are in contact with each other.

In other examples, the term “over” may mean that one component or material is positioned indirectly on another component or material. By “indirectly on”, it is meant that a gap or an additional component or material may be positioned between the two components or materials.

As used herein, the term “PAZAM” may be a hydrogel containing reactive azide group which performs a “click” reaction with the norbornene group.

As used herein, the term “PEG” (polyethylene glycol) may be a polyether compound with a structure of H—(O—CH₂—CH₂)ₙ—OH.

As used herein, the terms “poly-azide,” “poly-azido,” “multi-azide” and “multi-azido” are synonymous and used interchangeably to refer to a molecule having two or more azide or azido functionalities.

As used herein, the term “polymerase” is intended to be consistent with its use in the art and includes, for example, an enzyme that produces a complementary replicate of a nucleic acid molecule using the nucleic acid as a template strand. DNA polymerases may bind to the template strand and then move down the template strand sequentially adding nucleotides to the free hydroxyl group at the 3′ end of a growing strand of nucleic acid. DNA polymerases synthesize complementary DNA molecules from DNA templates, and ribonucleic acid (RNA) polymerases synthesize RNA molecules from DNA templates (transcription). Polymerases can use a short RNA or DNA strand, called a primer, to begin strand growth. Some polymerases can displace the strand upstream of the site where they are adding bases to a chain. Such polymerases are said to be strand displacing, meaning they have an activity that removes a complementary strand from a template strand being read by the polymerase. Example polymerases having strand displacing activity include, for example, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of a primer set may be modified to allow a coupling reaction with a functional group of one of the orthogonal polymers. The primer length can be any number of bases long and can include a variety of non-natural nucleotides. In an example, the sequencing primer is a short strand, ranging from 10 to 60 bases, or from 20 to 40 bases.

The term “qPCR” refers to a real-time polymerase chain reaction which is a laboratory technique of molecular biology based on the polymerase chain reaction (PCR). The qPCR may monitor the amplification of a targeted DNA molecule during the PCR (e.g., in real time), not at its end, as in conventional PCR.

As used herein, the term “rate,” when used in reference to diffusion, seeding, amplification, capture or other chemical processes, is intended to be consistent with its meaning in chemical kinetics and biochemical kinetics. Rates for two processes can be compared with respect to maximum rates (e.g., at saturation), pre-steady state rates (e.g., prior to equilibrium), kinetic rate constants, or other measures known in the art. In particular examples, a rate for a particular process can be determined with respect to the total time for completion of the process or a time interval between rates. For example, an amplification rate can be determined with respect to the time taken for amplification to be complete. However, a rate for a particular process need not be determined with respect to the total time for completion of the process.

As used herein, the term “recombinase” is intended to be consistent with its use in the art and includes, for example, RecA protein, the T4 uvsX protein, any homologous protein or protein complex from any phyla, or functional variants thereof. Eukaryotic RecA homologues are generally named Rad51 after the first member of this group to be identified. Other non-homologous recombinases may be utilized in place of RecA, for example, RecT or RecO.

The term “substrate” refers to a structure upon which various components of the flow cell (e.g., polymeric hydrogel, primers, etc.) may be added. The substrate may be a wafer, a panel, a rectangular sheet, a die, or any other suitable configuration. The substrate is generally rigid and is insoluble in an aqueous liquid. The substrate may be a single layer structure, or a multi-layered structure (e.g., including a support and a patterned material on the support). Examples of suitable substrates will be described further herein.

The term “TdT” (terminal deoxynucleotidyl transferase) refers to a specialized DNA polymerase expressed in immature, pre-B, pre-T lymphoid cells, and acute lymphoblastic leukemia/lymphoma cells. TdT adds N-nucleotides to the V, D, and J exons of the TCR and BCR genes during antibody gene recombination, enabling the phenomenon of junctional diversity.

The present disclosure provides methods and compositions for flow-cell based sequencing of clonal populations of a nucleic acid library individually clustered on a bead substrate, including complimentary reagents for functionalization of a flow cell surface for selective capture, in situ enrichment, imaging, and traceless release of clustered beads in a sequencing cycle.

The present disclosure provides methods for performing analysis on target nucleic acids that include one or more of (1) clustering, in a solution or suspension, a clonal amplicon population from each of a plurality of nucleic acid targets seeded on an array of bead substrates, wherein a desired portion of each clonal amplicon population is conjugated with a capture agent, and wherein clustering results in a pool of clustered bead substrates; (2) providing a flow cell substrate functionalized with a coating of binding partners on at least a portion of the substrate, wherein the binding partner has a high affinity for binding the capture agent; (3) loading the pool of clustered bead substrates onto the flow cell; (4) immobilizing at least a portion of the bead substrates onto the flow cell substrate via interactions between the binding agents of the clustered beads substrates and binding partners of the functionalized substrate of the flow cell; (5) performing sequence analysis on the immobilized bead substrates, (6) introducing a cleaving reagent to remove the immobilized substrates at the end of an analysis cycle, wherein the cleaving reagent dissociates the orthogonal complex under conditions that substantially conserve functionality of the coating of streptavidin moieties on the flow cell surface for multi-cycle use.

Also disclosed are methods that include one or more of (1) mixing a first reagent mixture with an amount of double stranded target nucleic acids within a reservoir to define a first solution, wherein the 5′ and 3′ ends of target nucleic acids are ligated with respective first and second adapters, and wherein the first reagent mixture comprises nucleoside triphosphates (NTPs), one or more replication enzymes, and adapter primers, wherein the adaptor primers include a primer mix of second adaptor primers and analogs primers conjugated with a capture agent in a desired ratio; (2) mixing the first solution with a second solution (or suspension) comprising primer grafted beads, wherein each primer is configured to hybridize to the first adapter of the target nucleic acids; (3) clustering to produce a pool of clustered beads, wherein target nucleic acids are seeded onto primer grafted beads and amplified to form clonal populations, wherein the rate of amplification rate preferably exceeds the rate at which target nucleic acids are seeded, and wherein each clonal population comprises amplicons synthesized with either a second adaptor primer or the primer analog conjugated with a capture agent, wherein the proportion of amplicons synthesized with the capture agent conjugate corresponds to the desired ratio of the primer mix, and wherein clustering yields a bead pool comprising well-clustered beads characterized by high occupancy of amplicons synthesized with the capture agent conjugate, poorly clustered beads characterized by low occupancy of amplicons synthesized with the capture agent conjugate relative to well-clustered beads, and unclustered beads lacking amplicons synthesized with the capture agent conjugate; (4) loading the bead pool is onto a flow cell substrate at least a portion of which is functionalized with a coating of binding partners that capture agents with high affinity such that well-clustered beads of the bead pool are selectively captured on the functionalized surface of the flow cell, which enables sorting of well-clustered beads from poorly clustered and unclustered beads from the bead pool; and (5) enriching the bead pool by removing poorly clustered and unclustered beads from the functionalized substrate of the flow cell.

Beads or particle architectures for use with the various embodiments described herein may including beads or particle architectures used for primer grafting, library seeding, and clustering and may be magnetic or non-magnetic micro- or nanoparticles composed of silica with or without a magnetic core, or polymer with or without a protein coating (e.g., neutravidin or streptavidin), or another polymer or hydrogel. The ideal range of hydrodynamic diameters of the bead may fall into any number of micro- or nano-scale ranges. Thus, for example, in certain embodiments bead diameter can range between 5 and 750 μm, including 25-700 μm, 50-650 μm, 75-600 μm, 100 to 550 μm, 150 to 500 μm, 300 to 450 μm. The surface of beads or particle architectures may be functionalized in a number of ways. For example, the surface may be functionalized with silanes and/or hydrogels/polymers (e.g., PAZAM) and coated with oligos or other chemical moieties such as azides, alkynes, DBCO, silanes, biotin, streptavidin, or neutravidin. The bead may be comprised of a silica bead or core-shell bead with a magnetic core and a silica shell. The bead may functionalize with norbornene, then react with PAZAM. Following PAZAM functionalization, alkyne-containing oligos may be further “clicked” onto the PAZAM surface to form a covalently modified DNA-coated (grafted) particle/bead. As discussed in more detail below, grafted particles may be clustered using kinetic exclusion with an ExAmp formulation (e.g., Ras6T).

A clonal population of a targeted nucleic acid of the present disclosure can be made using a method that exploits kinetic exclusion to ensure homogeneity of clustered beads. Generating clonal populations on an array of bead substrates where bead substrates are randomly seeded with target nucleic acids from a solution and copies of the target nucleic acid are generated in an amplification process to populate each bead surface to capacity ideally requires 1:1 seeding ratio to ensure the cluster results from amplification of only a single target molecule. However, because bead substrates are randomly seeded, a 1:1 seeding ratio is difficult to achieve because the variance of the number of targets molecules per bead tends to be stochastic and, thus, seeding events tend to occur along a Poisson distribution. Kinetic exclusion can occur when a process occurs at a sufficiently rapid rate to effectively exclude another event or process from occurring. Here, in accordance with the kinetic exclusion methods of the present disclosure, the seeding and amplification processes can proceed simultaneously under conditions where the amplification rate exceeds the rate at which target molecules are seeded. As such, the relatively rapid rate at which copies are made on bead substrate that has been seeded by a first target nucleic acid will effectively exclude a second nucleic acid from seeding the same substrate for amplification. Additional kinetic exclusion methods for amplifying nucleic acid libraries are described in US 2016/0053310 A1, which is incorporated herein by reference in its entirety.

Kinetic exclusion can exploit a relatively slow rate for making a first copy of a target nucleic acid vs. a relatively rapid rate for making subsequent copies of the target nucleic acid or of the first copy. In the example of the previous paragraph, kinetic exclusion occurs due to the relatively slow rate of target nucleic acid seeding (e.g., relatively slow diffusion or transport) vs. the relatively rapid rate at which amplification occurs to fill the bead substrate with copies of the nucleic acid seed. In another example, kinetic exclusion can occur due to a delay in the formation of a first copy of a target nucleic acid that has seeded a bead substrate (e.g., delayed or slow activation) versus the relatively rapid rate at which subsequent copies are made to fill the same bead substrate. In this example, an individual bead may have been seeded with several different target nucleic acids (e.g., several target nucleic acids can be present on the bead substrate prior to amplification). However, first copy formation for any given target nucleic acid can be activated randomly such that the average rate of first copy formation is relatively slow compared to the rate at which subsequent copies are generated. In this case, although an individual bead may have been seeded with several different target nucleic acids, kinetic exclusion will allow only one of those target nucleic acids to be amplified. More specifically, once a first target nucleic acid has been activated for amplification, the bead substrate will rapidly fill to capacity with its copies, thereby preventing copies of a second target nucleic acid from being made on the bead.

In some examples of the methods set forth herein, it is desirable to use a population of target nucleic acids that is double stranded. It has been surprisingly observed that amplicon formation on a bead-based array under kinetic exclusion conditions is efficient for double stranded target nucleic acids. For example, a plurality of bead substrates having clonal populations of amplicons can be more efficiently produced from double stranded target nucleic acids (compared to single stranded target nucleic acids at the same concentration) in the presence of recombinase and single stranded binding protein. Moreover, as described in more detail below, double stranded targets, particularly DNA targets, are amenable to 3′end click chemistry tailing for modification of dsDNA-coated beads. Nevertheless, it will be understood that single stranded target nucleic acids can be used in some examples of the methods set forth herein.

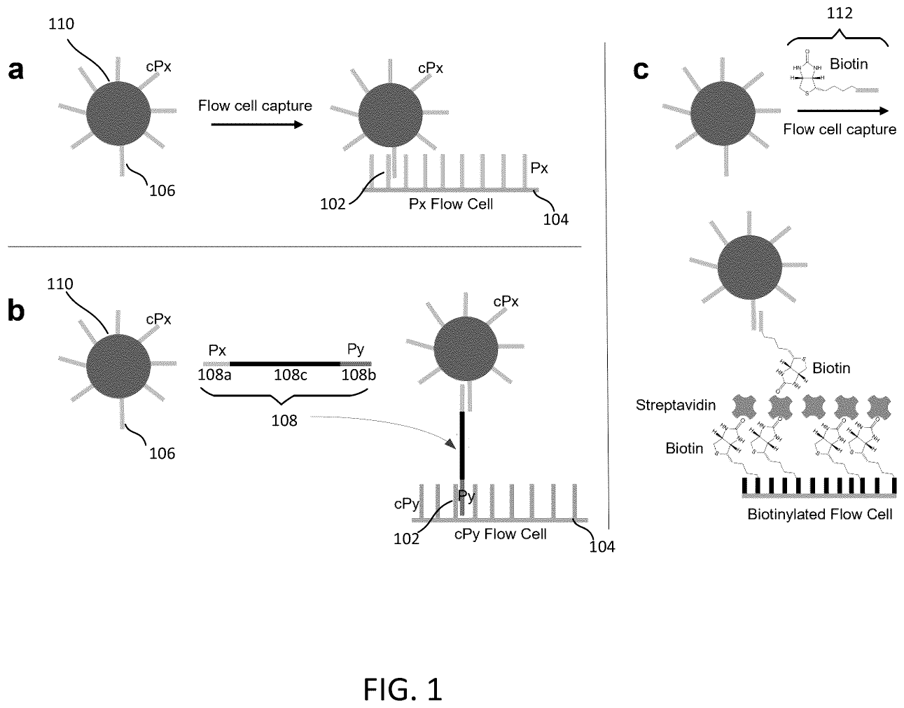

The reagents for use with various embodiments set forth herein may include a capture agent and binding partner, one arranged on the clustered beads and the other on the flow cell surface, and the attachment between the capture agent and binding partner may be direct or made through an intermediate structure. For example, the binding agent may be a nucleic acid strand bound to the cell flow surface that directly hybridizes to a sequence of one or more clonal populations. Capture nucleic acids having a universal capture sequence complementary to an adapter sequence common across clonal populations are particularly apt. For example, as shown in the illustration of FIG. 1(a), a capture nucleic acid 102 bound to the flow cell surface 104 may directly hybridize an amplicon 106 bound to clustered bead 110 via P5/cP5 or P7/cP7 complementarity.

Capture nucleic acids contemplated herein may also bind amplicons indirectly through an intermediate structure. For example, FIG. 1(b) shows an example of a bridge hybridization approach in which a capture nucleic acid 102 indirectly binds amplicon 106 via nucleic acid bridge molecule 108 that spans the region between the flow cell 104 and the clustered bead 110 and has, at one end, a sequence 108b complementary to a sequence of the capture nucleic acid 102 on the flow cell surface 104 and, at the other end, a sequence 108a complementary to a sequence on the amplicon(s) 106. Preferably the complementary sequences the respective ends of the bridge molecule 108 each hybridize to a different adapter on the respective capture nucleic acids and amplicons. In the example of FIG. 1(b), the bridge molecule 108 may link the capture nucleic acid 102 and an amplicon 106 through respective P5/cP5 and P7/cP7 complementarity. One advantage of bridge hybridization for use with embodiments herein is that it allows for incorporation of a cleavable linker 108c that may be triggered photochemically (via photocleavable groups such as orthonitrobenzyl or related groups) or enzymatically (restriction sites or uracil cleavage) to selectively release the clustered beads from the flow cell surface at the completion of a sequencing cycle.

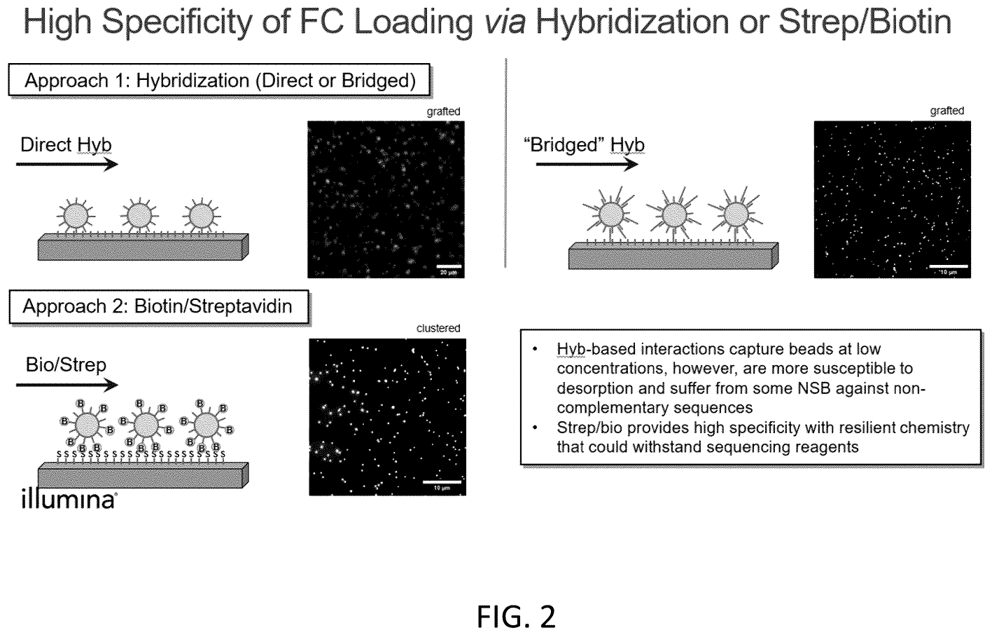

Complimentary reagent chemistry used for bead capture is preferably orthogonal to the hybridization-based chemistry used to functionalize the bead substrate for amplification such that captured beads may be selectively cleaved under conditions that conserve flow cell surface chemistry for repeated reuse. In preferred embodiments, bead capture is accomplished through biotin-avidin interaction. The biotin ligand and avidin protein are particularly apt because they bind with high affinity to form a stable complex that can withstand the pH, temperatures, solvents, and denaturing agents involved in flow cell chemistry. Moreover, biotin-avidin interaction provides higher specificity of flow cell loading compared to hybridization-based interactions, as demonstrated in Example I (using a streptavidin moiety) and the accompanying fluorescence images presented in FIG. 2. Importantly, the biotin-avidin complex can be disassociated with cleaving agents under conditions that do not interfere with other flow cell surface chemistry and, perhaps more importantly, biotin and avidin moieties retain their high binding affinity for complex formation after dissociation. Accordingly, a flow cell substrate coated with either a biotin or avidin layer retains bead binding functionality throughout multi-cycle use.

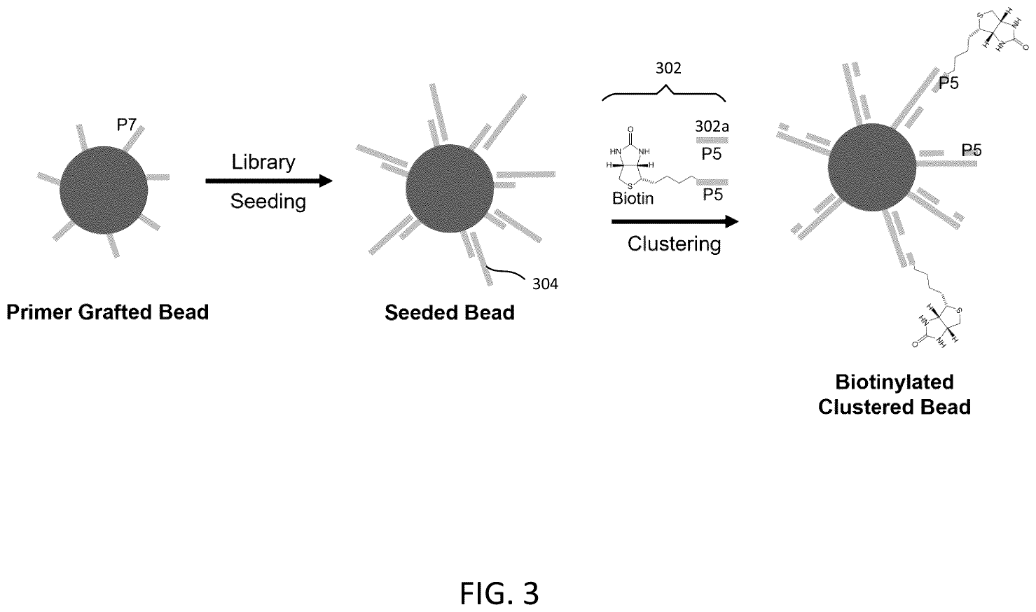

For some embodiments, an example of which is shown in FIG. 1(c), a biotin capture agent 112 is conjugated to one or more amplicons of a clonal population through hybridization of a biotinylated complimentary oligonucleotide. Preferably, in other embodiments, an example of which is shown in FIG. 3, a biotin capture agent 302 is conjugated to one or more amplicons 304 of a clonal population through hybridization using a covalent P5 linkage 302a. According to another strategy, biotin moiety may be selectively, site specifically conjugated to an amplicon during clustering through incorporation of a biotinylated adaptor primer included in a reagent mixture of biotinylated and non-biotinylated adaptor primers. According to this strategy, a bead is first grafted with a primer such as P7, and then clustered using Ras6T or another ExAmp reagent in an exclusion amplification process. P5 is used as an in-solution primer and mixed with its biotinylated analog in the desired ratio such as 10%, 20%, or 30% (or even up to 100%) of biotinylated primer to primer mixture. This adds a P5 into the resulting amplicons, wherein the desired proportion of biotinylated amplicons in a given clonal population may be obtained using a proportionate ratio of P5 primer to biotinylated analog in the reagent mixture.

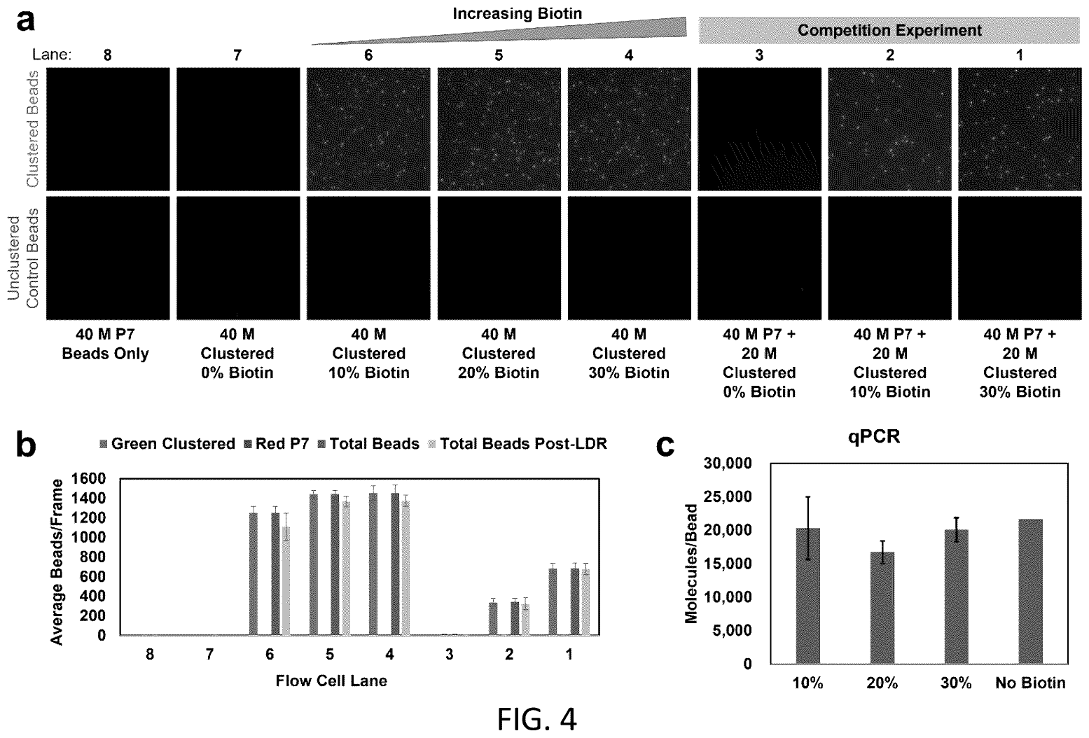

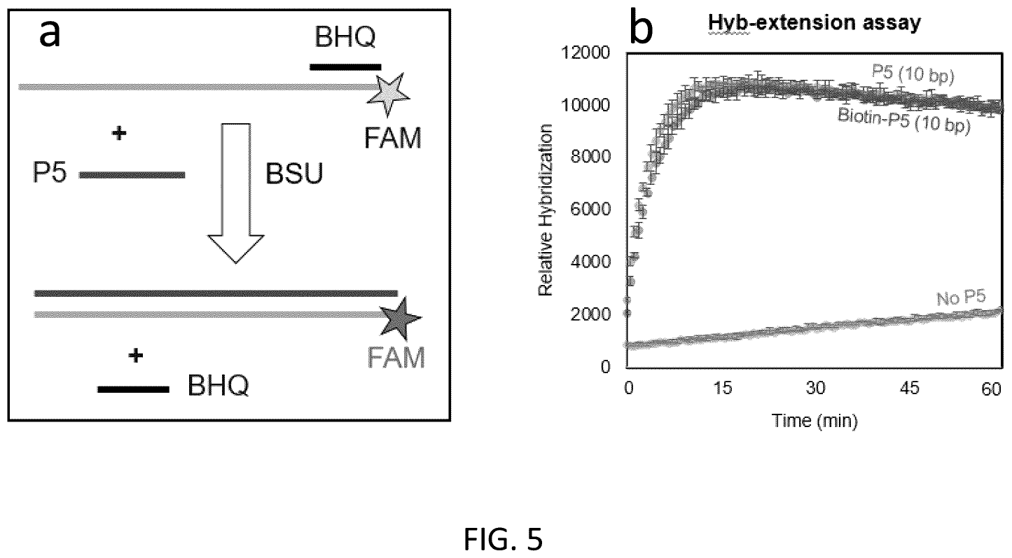

As demonstrated in Example II and the accompanying fluorescence images and graphical data presented in FIG. 4, discussed in more detail below, clustered beads biotinylated with a 20% biotinylated primer reagent mixture were captured with greater efficiently than beads biotinylated with a 10% mixture, yet increased biotinylation had no negative effect on qPCR yields. Moreover, as demonstrated in Example III and the accompanying scatter plot (b) of relative hybridization presented in FIG. 5, discussed in more detail below, incorporation of biotin via biotinylated P5 primer has no significant impact on hybridization/extension chemistries during sequencing operation.

Notably, other biotin binding proteins may be used as a complementary reagent in addition to avidin. For example, streptavidin and neutravidin each have sufficiently high binding affinity for biotin for use as a complimentary reagent for flow cell capture of biotinylated, clustered beads. In addition, ligands suitable for use as a complementary reagent to avidin include both natural biotin, its biological precursors, and synthesized biotin derivatives, e.g., biotin dimers, or biotin-PEG conjugates. Avidin-based ligands characterized by high binding specificity and relatively low binding affinity are particularly apt. These ligands form readily reversible complexes with the avidin (or streptavidin or neutravidin) moiety, which may be disassociated under physiological conditions by biotin. These ligands include, e.g., biotin analogs d-desthiobiotin and iminobiotin. (See Analytical Biochemistry 308 (2002) 343-357.) Other complementary partners contemplated herein include 1, 2- and/or 1, 3-diols substrates and boronic acid or boronic acid analog complements reversibly bound via ester exchange reaction; conjugated diene and dienophiles reversibly bound via cycloaddition (i.e., Diels-Alder reaction); peptide conjugates bound through reversible native chemical ligation (NCL); and click chemistry conjugates, including, e.g., triazolinedione (TAD) click conjugation, maleimide-based Diels-Alder click conjugation.

In a preferred embodiment, clustered beads are selectively, site-specifically chemically modified during the clustering process to incorporate a biotin which can bind to complimentary chemistry functionalized on a flow cell. The bead is first grafted with a primer such as P7, and then clustered using Ras6T or another ExAmp reagent during a process known as non-bridging clustering. P5 is used as an in-solution primer and mixed with its biotinylated analog in the desired ratio such as 10%, 20%, or 30% (or even up to 100%) of biotinylated primer to primer mixture. This incorporates a P5 into the clusters, and the amount of biotinylated primer added determines the amount of biotin in the final product, with increasing amounts of biotin with increasing percentage of biotinylated P5 until saturation is reached.

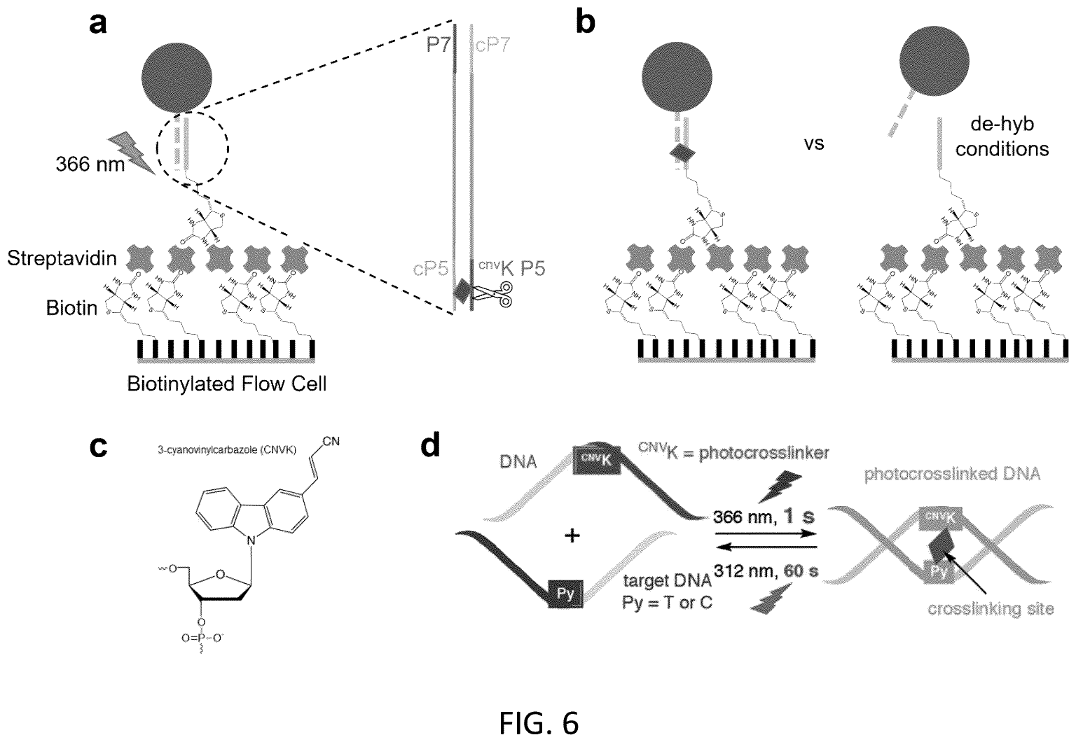

As discussed, a plurality of bead substrates having clonal populations of amplicons can be more efficiently produced from double stranded target nucleic acids (compared to single stranded target nucleic acids at the same concentration) in the presence of recombinase and single stranded binding protein. Moreover, biotin incorporation into the clustering process by way of a biotinylated in-solution P5 primer creates an effective method for selective capture and enrichment of clustered beads comprising biotin-conjugated double stranded amplicons, especially on a polymer-coated flow cell surface which may be influenced by hydrogen bonding or other polymer-polymer interactions. However, biotin-conjugated double stranded amplicons may be subject to de-hybridization conditions on the flow cell surface during subsequent cycle processes. Accordingly, certain embodiments of the methods herein include cross-linking biotinylated double stranded amplicons using a photoactive moiety to prevent denaturing and loss of the biotin moiety. The photoactive moieties for use with the embodiments may include an appropriate vinylcarbazole crosslinker, e.g., 3-cyanovinylcarbazole nucleoside (CNVK) (the structural formula of which is depicted in FIG. 6(c)), which may be incorporated in one of the strands during the clustering process and then cross-linked to the complementary strand using a single wavelength trigger as described in Org. Lett. 2008, 10, 15, 3227-3230, which is incorporated herein in its entirety. In the example method of FIG. 6(a), CNVK is incorporated into P5 strands that are biotinylated at the 5′ end using a CNVK-linked biotinylated P5 primer doped into the clustering primer mix. As illustrated in FIG. 6(d), CNVK incorporated into P5 strands can then be photo cross-linked upon UV (366 nm) irradiation. The kinetics of these cross-linking reactions are very fast, and the reaction typically nears completion within several seconds. After photo-crosslinking, the biotin functionalized P5 strands are covalently attached to the cP5 strands with enhanced chemically stability to preserve the biotinylated cross-linked double strand under the following processing conditions: de-hybridization (as illustrated in FIG. 6(b), hybridization of sequencing primers, and SBS chemistry. The proposed CNVK nucleoside is commercially available (Glen Research, Sterling, VA) and its crosslinking reaction is reversible upon 312 nm irradiation. Other cross-linking reactions contemplated herein include, e.g., oxime-formation click chemistry, in which an oxime bond is formed via condensation of an aminooxy group incorporated on a 3′-ONH2 terminated nucleoside at the end of one strand with a di-aldehyde as cross-linker.

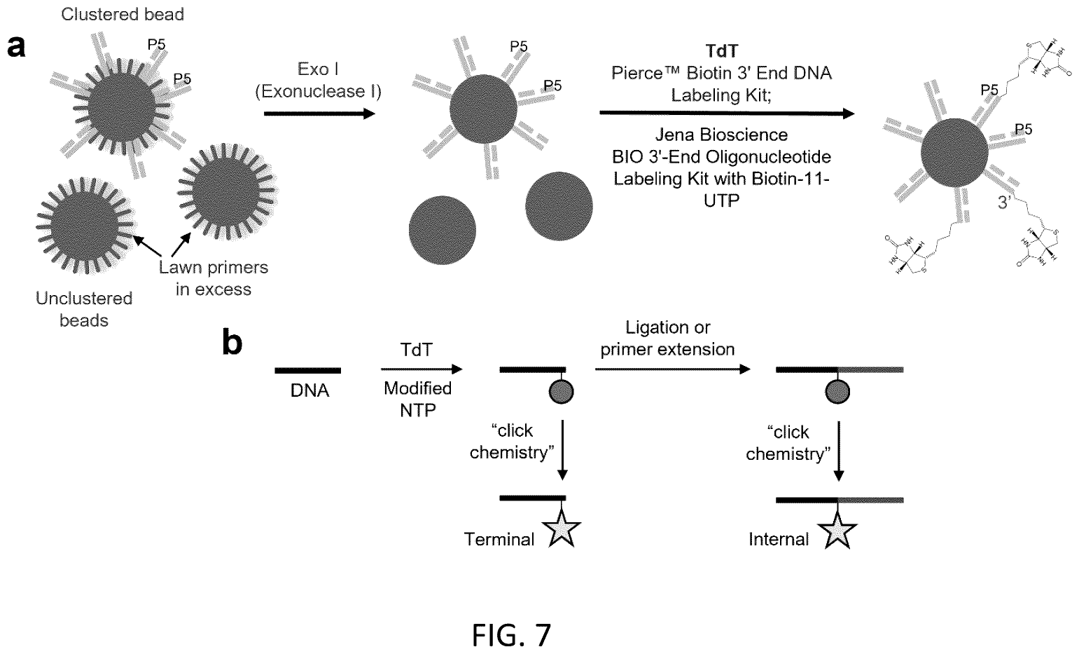

In certain embodiments, as an alternative to direct incorporation of biotin conjugated to the P5 adaptor during the clustering process, biotin may be added at the 3′ end of the double stranded amplicon using the enzyme terminal deoxynucleotidyl transferase (TdT) in accordance with methods described in Nucleic Acids Res. 2015, 43, 17, e110, which is incorporated herein by reference in its entirety. TdT is capable of adding a wide variety of modified NTPs which can contain clickable groups such as azides or linear or cyclic alkynes. As described previously, the current clustering process results in a mixture of unclustered and clustered beads, but this chemical modification workflow can separate the two populations. In accordance with the example method illustrated in FIG. 7(a), a pool of beads are first treated with Exonuclease 1 (Exo 1) to remove excess lawn primers, followed by TdT and modified NTPs such as biotinylated NTPs. As shown in FIG. 7(b), click chemistry workflow is amenable to a variety of modified NTPs including those with “clickable” groups (such as azide and alkyne) and can also be followed by ligation or primer extension to create internal modifications. This workflow enables enrichment of clustered beads onto a flow cell by selective incorporation of biotin or clickable groups into clustered beads.

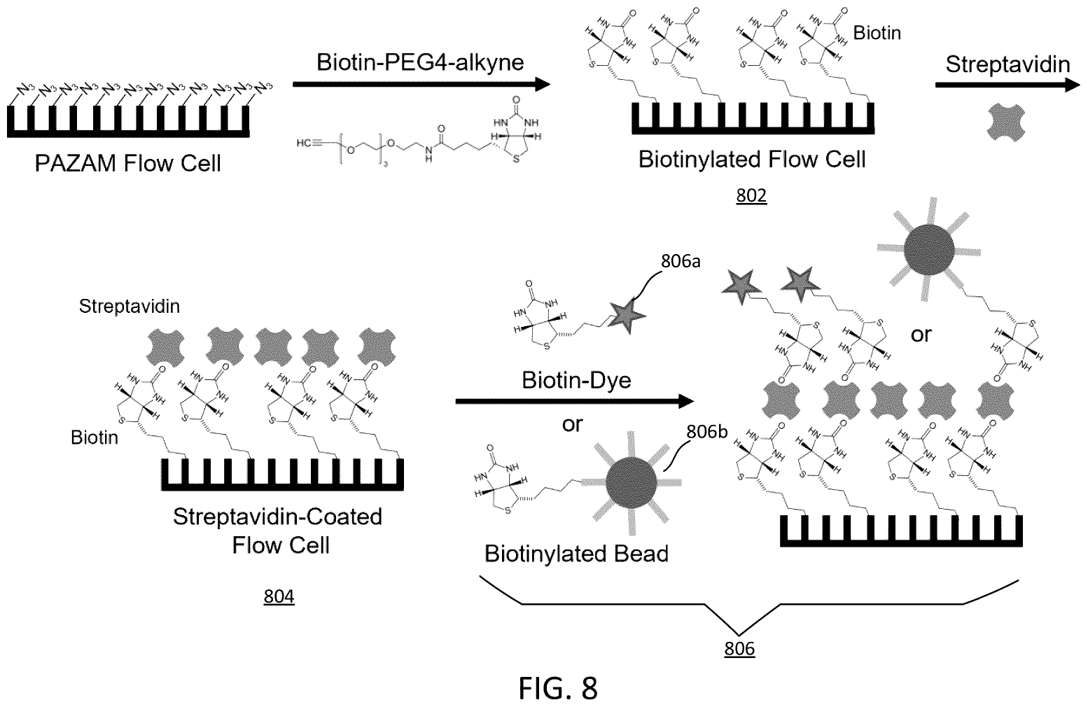

The flow cell for use with the embodiments herein may comprise a glass substrate functionalized with any of the following-silane layer, NIL resin, PAZAM polymer, or variant of PAZAM polymer or hydrogel. The capture moieties may be on the surface of the PAZAM or other polymer/hydrogel, directly on the glass or NIL resin, or may be incorporated into the silane layer, for direct capture in a polymer-free approach. PAZAM contains azide groups which are used for click reactions with alkynes (strained or unstrained) such as biotin-PEG4-alkyne. The PEG or other solubilizing group allows biotin to dissolve in aqueous solutions. In accordance with an example method shown in FIG. 8, after clicking on to the PAZAM layer at 802, the biotin efficiently reacts with free streptavidin at room temperature, creating a layer of streptavidin on the flow cell at 804. Due to the multi-binding pocket nature of streptavidin, it is then able to react further with additional biotin moieties at 806. This additional biotin can be introduced as a small molecule such as a biotin-dye 806a which can be used for QC purposes, or as a biotin-incorporated bead 806b, as further shown in FIGS. 4a and 4b.

A more detailed example of kinetic exclusion via differential rates of amplicon formation follows. A bead substrate can include three subpopulations of primers attached thereto. The first subpopulation of primers functions to capture a target nucleic acid (via a capture sequence) and as a primer for first amplicon formation. The first subpopulation of primers is reversibly blocked from extension, for example, via a dideoxy nucleotide at the 3′ end. The second subpopulation of primers can have a P5 primer sequence and the third population of primers can have a P7 primer sequence. The primers of the first and second subpopulations do not include the dideoxy nucleotide and are therefore fully extension competent. Target nucleic acids can be constructed to include (from 5′ to 3′) a P7 primer binding sequence, one of several different target nucleotide sequences, a P5 primer binding sequence, and a capture sequence complement. Several different target nucleic acids can be hybridized to the first subpopulation of primers (via the capture sequences). The capture primers can then be converted to an extendible state, for example, by treatment with a polymerase under pyrophosphorolysis conditions (e.g., in the presence of excess pyrophosphate). Conditions can be used where, on average, only one of the capture primers will be converted to an extendible form during the time period in which subsequent amplicons are produced to fill the bead. Thus, although several potentially contaminating target nucleic acids may be present at an individual bead substrate, kinetic exclusion will result in amplicon formation from only one of the target nucleic acids, thereby creating a clonal population of amplicons on the bead.

Any of a variety of temporarily non-extendible primers can be used in a method set forth herein along with respective techniques and reagents for converting those primers to an extendible state. The example above describes use of a dideoxy nucleotide that is removed by pyrophosphorolysis. Other non-extendible nucleotides can be present on a primer and removed by pyrophosphorolysis. Furthermore, dideoxy nucleotides or other non-extendible nucleotides can be removed via other known techniques including, for example, exonuclease activity of a polymerase or other appropriate enzyme. In other examples, a primer can include a reversible terminator such as those used in terminator-based sequencing-by-synthesis methods. Examples of reversible terminators and techniques for their removal are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and U.S. Patent Publ. No. 2008/0108082, each of which is incorporated herein by reference in its entirety.

Although the use of differentially active primers to cause different rates of first amplicon and subsequent amplicon formation has been exemplified above for an example where target nucleic acids are present at bead substates prior to amplification, the method can also be carried out under conditions wherein the target nucleic acids are transported (e.g., via diffusion) to the bead substrates as amplification is occurring. Thus, kinetic exclusion can exploit both a relatively slow transport rate and a relatively slow production of first amplicon relative to subsequent amplicon formation. Thus, an amplification reaction set forth herein can be carried out such that target nucleic acids are transported from solution to bead substrates simultaneously with (i) the producing of a first amplicon, and (ii) the producing of the subsequent amplicons at other bead substrates of the array. In particular examples, the average rate at which the subsequent amplicons are generated at the amplification sites can exceed the average rate at which the target nucleic acids are transported from the solution to the bead substrates. In some cases, a sufficient number of amplicons can be generated from a single target nucleic acid at an individual substrate to fill the capacity of the respective substrate. The rate at which amplicons are generated to fill the capacity of respective bead substrate can, for example, exceed the rate at which the individual target nucleic acids are transported from the solution to the substrates.

Other techniques may be employed as a complement to ExAmp or other contemplated amplification techniques to better ensure homogeneity of clustered beads. For example, in certain method embodiments, a low-density (sub-Poisson) seeding technique may be performed prior to or, in the case of ExAmp, simultaneous with, target molecule amplification. Low density (sub-Poisson) seeding is performed by adding target molecules at low concentration to a bead solution or suspension having a large excess of beads. Doing so effectively narrows the probability distribution of stochastic variance and skews the distribution mean toward a 1:1 ratio, and, thus, the likelihood of two (or more) target molecules seeding onto the same bead is significantly reduced. For non-exclusion amplification methods in particular, after low density seeding is completed, any excess target molecules can be washed off the beads prior to amplification.

An amplification reagent that is used in a method set forth herein is preferably capable of rapidly making copies of target nucleic acids on the bead substrate. One or more amplification reagents used in a method of the present disclosure will include a polymerase and nucleoside triphosphates (NTPs). Any of a variety of polymerases known in the art can be used, but in some examples it may be desirable to use a polymerase that is exonuclease negative. The NTPs can be deoxyribonucleoside triphosphates (dNTPs) for examples where DNA copies are made. The four native species of dNTPs, including dATP, dTTP, dGTP and dCTP, may be present in a DNA amplification reagent; however, analogs can be used if desired. The NTPs can be ribonucleoside triphosphates (rNTPs) for examples where RNA copies are made. The four native species of rNTPs, including rATP, rUTP, rGTP and rCTP, may be present in an RNA amplification reagent; however, analogs can be used if desired.

An amplification reagent can include further components that facilitate amplicon formation and in some cases increase the rate of amplicon formation. An example is a recombinase. A recombinase can facilitate amplicon formation by allowing repeated invasion/extension. More specifically, a recombinase can facilitate invasion of a target nucleic acid by the polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single stranded binding (SSB) protein is particularly useful as SSB can further facilitate amplification. Example formulations for recombinase-facilitated amplification include those sold commercially as TWISTAMP® kits by TwistDx (Cambridge, UK). Useful components of recombinase-facilitated amplification reagent and reaction conditions are set forth in U.S. Pat. Nos. 5,223,414 and 7,399,590, each of which is incorporated herein by reference in its entirety.

Another example of a component that can be included in an amplification reagent to facilitate amplicon formation, and in some cases to increase the rate of amplicon formation is a helicase. Helicase can facilitate amplicon formation by allowing a chain reaction of amplicon formation. The process can occur more rapidly than standard PCR since a denaturation cycle (e.g., via heating or chemical denaturation) is not required. As such, helicase-facilitated amplification can be carried out isothermally. A mixture of helicase and single stranded binding (SSB) protein is particularly useful as SSB can further facilitate amplification. Example formulations for helicase-facilitated amplification include those sold commercially as ISOAMP® kits from Biohelix (Beverly, Mass.). Further, examples of useful formulations that include a helicase protein are described in U.S. Pat. Nos. 7,399,590 and 7,829,284, each of which is incorporated herein by reference in its entirety.

Yet another example of a component that can be included in an amplification reagent to facilitate amplicon formation, and in some cases increase the rate of amplicon formation, is an origin binding protein.

The rate at which an amplification reaction occurs can be increased by increasing the concentration or amount of one or more of the active components of an amplification reaction. For example, the amount or concentration of polymerase, nucleoside triphosphates, primers, recombinase, helicase or SSB can be increased to increase the amplification rate. In some cases, the one or more active components of an amplification reaction that are increased in amount or concentration (or otherwise manipulated in a method set forth herein) are non-nucleic acid components of the amplification reaction.

The amplification rate can also be increased in a method set forth herein by adjusting the temperature. For example, the rate of amplification at one or more amplification sites can be increased by increasing the temperature at the site(s) up to a maximum temperature where reaction rate declines due to denaturation or other adverse events. Optimal or desired temperatures can be determined from known properties of the amplification components in use or empirically for a given amplification reaction mixture. Properties of primers used for amplification can also be adjusted to increase amplification rate. For example, the sequence and/or length of primers can be adjusted. Such adjustments can be made based on a priori predictions of primer melting temperature (Tm) or empirically.

Another option for increasing the rate of amplification at an amplification site is to increase the concentration of one or more active components of the amplification reaction. Affinity tags can be used as well. For example, any of a variety of ligands or receptors known in the art, such as those set forth herein as examples of capture agents, can be used as affinity tags for a component of an amplification reaction. As is the case for capture agents used for nucleic acids, a bead substrate can include a binding partner for an affinity tag of an amplification component. Thus, the local concentration of the affinity tagged amplification component can be increased due to interaction with the appropriate partner at the site of amplification.

The rate at which an amplification reaction occurs can be increased by increasing the activity of one or more amplification reagents. For example, a cofactor that increases the extension rate of a polymerase can be added to a reaction where the polymerase is in use. In some examples, metal cofactors, such as magnesium, zinc, or manganese, can be added to a polymerase reaction, or, in other examples, betaine can be added.

A method set forth herein can use any of a variety of amplification techniques. Example techniques that can be used include, but are not limited to, polymerase chain reaction (PCR), rolling circle amplification (RCA), multiple displacement amplification (MDA), or random prime amplification (RPA). In some examples the amplification can be carried out in the bead solution or suspension, for example, when the bead substrates are capable of containing amplicons in a volume having a desired capacity. In alternative embodiments, seeding is carried out in the bead solution or suspension, and amplification is carried out on the bead substrates after being immobilized on the flow cell. Whichever the case, an amplification technique used under conditions of kinetic exclusion in a method of the present disclosure, preferably, may be carried out on solid phase bead substrate. For example, one or more primers used for amplification can be attached to a solid phase on the bead substrate. In PCR examples, one or both of the primers used for amplification can be attached to a solid phase. Formats that utilize two species of primer attached to the surface are often referred to as bridge amplification because double stranded amplicons form a bridge-like structure between the two surface-attached primers that flank the template sequence that has been copied. Example reagents and conditions that can be used for bridge amplification are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420, each of which is incorporated herein by reference in its entirety. Solid-phase PCR amplification can also be carried out with one of the amplification primers attached to a solid support and the second primer in solution. An example format that uses a combination of a surface attached primer and soluble primer is emulsion PCR as described, for example, in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference in its entirety. Emulsion PCR is illustrative of the format and it will be understood that for purposes of the methods set forth herein the use of an emulsion is optional, and indeed, for several examples, an emulsion is not used. The above-exemplified PCR techniques can be modified for non-cyclic amplification (e.g., isothermal amplification) using components exemplified elsewhere herein for facilitating or increasing the rate of amplification. Accordingly, the above-exemplified PCR techniques can be used under kinetic exclusion conditions.

RCA techniques can be modified for use in a method of the present disclosure. Example components that can be used in an RCA reaction and principles by which RCA produces amplicons are described, for example, in Lizardi et al., Nat. Genet. 19:225-232 (1998) and U.S. Patent Publ. No. 2007/0099208 A1, each of which is incorporated herein by reference in its entirety. Primers used for RCA can be in solution or attached to a solid support surface at a bead substrate. The RCA techniques exemplified in the above references can be modified in accordance with teaching herein, for example, to increase the rate of amplification to suit particular applications. Thus, RCA techniques can be used under kinetic exclusion conditions.

MDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference in its entirety. Primers used for MIDA can be in solution or attached to a solid support surface at a bead substrate. The MDA techniques exemplified in the above references can be modified in accordance with teaching herein, for example, to increase the rate of amplification to suit particular applications. Accordingly, MDA techniques can be used under kinetic exclusion conditions.

In particular examples, a combination of the above-exemplified amplification techniques can be used to make an array under kinetic exclusion conditions. For example, RCA and MDA can be used in a combination wherein RCA is used to generate a concatameric amplicon in solution (e.g., using solution-phase primers). The amplicon can then be used as a template for MDA using primers that are attached to a solid support surface on the bead substrate. In this example, amplicons produced after the combined RCA and MDA steps will be attached to the bead surface.

As exemplified with respect to several of the examples above, a method of the present disclosure need not use a cyclical amplification technique. For example, amplification of target nucleic acids can be carried out at amplification sites absent a denaturation cycle. Example denaturation cycles include introduction of chemical denaturants to an amplification reaction and/or increasing the temperature of an amplification reaction. Thus, amplifying of the target nucleic acids need not include replacing the amplification solution with a chemical reagent that denatures the target nucleic acids and the amplicons. Similarly, amplifying of the target nucleic acids need not include heating the solution to a temperature that denatures the target nucleic acids and the amplicons. Accordingly, amplifying of target nucleic acids on bead substrates can be carried out isothermally for the duration of a method set forth herein. Indeed, an amplification method set forth herein can occur without one or more cyclic manipulations that are carried out for some amplification techniques under standard conditions. Furthermore, in some standard solid phase amplification techniques, a wash is carried out after target nucleic acids are loaded onto a substrate and before amplification is initiated. However, in examples of the present methods, a wash step need not be carried out between transport of target nucleic acids to bead substrates and amplification of the target nucleic acids. Instead, transport (e.g., via diffusion) and amplification are allowed to occur simultaneously to provide for kinetic exclusion.

In some examples it may be desirable to repeat an amplification cycle that occurs under kinetic exclusion conditions. Thus, although copies of a target nucleic acid can be made at an individual amplification site via kinetic exclusion amplification without cyclic manipulations involving chemical denaturants or heat applications, an array of bead substrates can be treated cyclically to increase the number of sites that contain amplicons and/or the number of amplicons at each site after each cycle of kinetic exclusion amplification. For example, a secondary amplification or boost may be performed after the initial exclusion amplification by dosing a fresh, target-less solution that includes reagents (e.g., active components) without target nucleic acids. The reagents in bead substrates e target-less solution may increase the number of amplicons in the clonal clusters on the array of beads. Due to the lack of target nucleic acids in the target-less solution, the reagent may not cause additional seeding of target nucleic acids on the bead substrate. In particular examples, the amplification conditions can be modified from one cycle to the next. For example, one or more of the conditions set forth above for altering the seeding rate or altering the rate of amplification can be adjusted between cycles.

In various of the disclosed embodiments the clustered beads solution (or suspension) is adapted to flow through a patterned (or structured) flow cell comprising microwells or a planar array. Using biotin-streptavidin complements for illustration, the microwell or planar array surfaces are functionalized with a streptavidin coating. As the beads diffuse in or across these functionalized surfaces of the flow cell well-clustered beads, characterized by high biotinylation, become immobilized, whereas unclustered or poorly clustered beads, characterized by low or no biotinylation, can be selectively washed away by a buffer, e.g., HT2 buffer.

A bead-based array of the present disclosure, for example, having been produced by a method set forth herein, can be used for any of a variety of applications. A particularly useful application is nucleic acid sequencing. One example is sequencing-by-synthesis (SBS). In SBS, extension of a nucleic acid primer along a nucleic acid template (e.g., a target nucleic acid or amplicon thereof) is monitored to determine the sequence of nucleotides in the template. The underlying chemical process can be polymerization (e.g., as catalyzed by a polymerase enzyme). In a particular polymerase-based SBS example, fluorescently labeled nucleotides are added to a primer (thereby extending the primer) in a template dependent fashion such that detection of the order and type of nucleotides added to the primer can be used to determine the sequence of the template. A plurality of different templates at different sites of an array set forth herein can be subjected to an SBS technique under conditions where events occurring for different templates can be distinguished due to their location in the array.

Flow cells provide a convenient format for capturing the bead-based arrays that are produced by the methods of the present disclosure and that is subjected to an SBS or other detection technique that involves repeated delivery of reagents in cycles. For example, to initiate a first SBS cycle, one or more labeled nucleotides, DNA polymerase, etc., can be flowed into/through a flow cell that houses the bead based array of nucleic acid templates. Those sites of an array where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for examples that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Example SBS procedures, fluidic systems and detection platforms that can be readily adapted for use with an array produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and U.S. Patent Publ. No. 2008/0108082, each of which is incorporated herein by reference in its entirety.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference in its entirety). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be used for application of pyrosequencing to arrays of the present disclosure are described, for example, in U.S. Pat. No. 9,096,899, U.S. Patent Publ. No. 2005/0191698 A1, U.S. Pat. Nos. 7,595,883, and 7,244,559, each of which is incorporated herein by reference in its entirety.

Sequencing-by-ligation reactions are also useful including, for example, those described in Shendure et al. Science 309:1728-1732 (2005); U.S. Pat. Nos. 5,599,675; and 5,750,341, each of which is incorporated herein by reference in its entirety. Some examples can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety. In both sequencing-by-ligation and sequencing-by-hybridization procedures, target nucleic acids (or amplicons thereof) that are present at sites of an array are subjected to repeated cycles of oligonucleotide delivery and detection. Fluidic systems for SBS methods as set forth herein or in references cited herein can be readily adapted for delivery of reagents for sequencing-by-ligation or sequencing-by-hybridization procedures. The oligonucleotides may be fluorescently labeled and detected using fluorescence detectors similar to those described with regard to SBS procedures herein or in references cited herein.

Some examples can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), each of which is incorporated herein by reference in its entirety.

Some SBS examples include detection of a proton released upon incorporation of a nucleotide into an extension product. For example, sequencing based on detection of released protons can use an electrical detector, and associated techniques are commercially available from Ion Torrent (Guilford, Conn., a Life Technologies subsidiary) or sequencing methods and systems are described in U.S. Patent Publ. No. 2009/0026082 A1; U.S. Patent Publ. No. 2009/0127589 A1; U.S. Patent Publ. No. 2010/0137143 A1; or U.S. Patent Publ. No. 2010/0282617 A1, each of which is incorporated herein by reference in its entirety. Methods set forth herein for amplifying target nucleic acids using kinetic exclusion can be readily applied to substrates used for detecting protons. More specifically, methods set forth herein can be used to produce clonal populations of amplicons at the sites of the arrays that are used to detect protons.

An advantage of the methods set forth herein is that they provide for rapid and efficient creation of bead-based arrays from any of a variety of nucleic acid libraries. Accordingly, the present disclosure provides integrated systems capable of making an array using one or more of the methods set forth herein and further capable of detecting nucleic acids on the arrays using techniques known in the art such as those exemplified above. Thus, an integrated system of the present disclosure can include fluidic components capable of delivering sequencing reagents to clustered beads immobilized on the flow cell such as pumps, valves, reservoirs, fluidic lines and the like. A particularly useful fluidic component is a flow cell. A flow cell can be configured and/or used in an integrated system to create an array of immobilized clustered beads of the present disclosure and to detect the array. Example flow cells are described, for example, in U.S. Patent Publ. No. 2010/0111768 A1 and U.S. Pat. No. 8,951,781, each of which is incorporated herein by reference in its entirety. As exemplified for flow cells, one or more of the fluidic components of an integrated system can be used for a detection method. Taking a nucleic acid sequencing example, one or more of the fluidic components of an integrated system can be used for the delivery of sequencing reagents in a sequencing method such as those exemplified above. Examples of integrated sequencing systems that are capable of sequencing bead-based arrays of nucleic acids, without limitation, the MISEQ® instrument platform (Illumina, Inc., San Diego, Calif.) and devices described in the U.S. Pat. No. 8,951,781 (referenced above).

A system capable of carrying out a detection method set forth herein, can include a system controller that is capable of executing a set of instructions to perform one or more methods, techniques or processes set forth herein. For example, the instructions can further direct the performance of steps for detecting nucleic acids using methods set forth previously herein. A useful system controller may include any processor-based or microprocessor-based system, including systems using microcontrollers, reduced instruction set computers (RISC), application specific integrated circuits (ASICs), field programmable gate array (FPGAs), logic circuits, and any other circuit or processor capable of executing functions described herein. A set of instructions for a system controller may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The software may be in various forms such as system software or application software. Further, the software may be in the form of a collection of separate programs, or a program module within a larger program or a portion of a program module. The software also may include modular programming in the form of object-oriented programming.

It will be understood that an array of the present disclosure, for example, having been produced by a method set forth herein, need not be used for a detection method. Rather, the array can be used to store a nucleic acid library. Accordingly, the array can be stored in a state that preserves the nucleic acids therein. For example, an array can be stored in a desiccated state, frozen state (e.g., in liquid nitrogen), or in a solution that is protective of nucleic acids. Alternatively, or additionally, the array can be used to replicate a nucleic acid library. For example, an array can be used to create replicate amplicons from one or more of the sites on the array.

Methods disclosed herein may be performed on a variety of different flow cell architectures, including a number of flow cells commercially available from Illumina, Inc. (San Diego, Calif.). In some examples, the flow cells can be configured with a functionalized substrate comprising an unstructured planar surface. In other examples, the flow cells can be configured with functionalized features in a surface. The features can be present in any of a variety of desired formats, including wells, pits, channels, ridges, raised regions, pegs, posts or the like. Example sites include wells that are present in substrates used for commercial sequencing platforms sold by 454 LifeSciences (a subsidiary of Roche, Basel Switzerland) or Ion Torrent (a subsidiary of Life Technologies, Carlsbad Calif.). Other substrates having wells include, for example, etched fiber optics and other substrates described in U.S. Pat. Nos. 6,266,459; 6,355,431; 6,770,441; 6,859,570; 6,210,891; 6,258,568; 6,274,320; U.S. Patent Publ. No. 2009/0026082 A1; U.S. Patent Publ. No. 2009/0127589 A1; U.S. Patent Publ. No. 2010/0137143 A1; U.S. Patent Publ. No. 2010/0282617 A1 or PCT Publication No. WO 00/63437, each of which is incorporated herein by reference in its entirety. In several cases, the substrates are exemplified in these references for applications that use beads in the wells. The well-containing substrates are particularly apt for immobilizing clustered beads of the disclosure into an array. In some examples, wells of a substrate can include gel material as set forth in U.S. Pat. No. 9,512,422, which is incorporated herein by reference in its entirety.

The functionalized features of the flow cell can be metal features on a non-metallic surface such as glass, plastic or other materials exemplified above. A metal layer can be deposited on a surface using methods known in the art, such as wet plasma etching, dry plasma etching, atomic layer deposition, ion beam etching, chemical vapor deposition, vacuum sputtering or the like. Any of a variety of commercial instruments can be used as appropriate including, for example, the FLEXAL®, OPAL™, IONFAB® 300plus, or OPTOFAB® 3000 systems (Oxford Instruments, UK). A metal layer can also be deposited by e-beam evaporation or sputtering as set forth in Thornton, Ann. Rev. Mater. Sci. 7:239-60 (1977), which is incorporated herein by reference in its entirety. Metal layer deposition techniques, such as those exemplified above, can be combined with photolithography techniques to create metal regions or patches on a surface. Example methods for combining metal layer deposition techniques and photolithography techniques are provided in U.S. Pat. No. 8,778,848, which is incorporated herein by reference in its entirety.

An array of functionalized features can appear as a grid of spots or patches. The features can be located in a repeating pattern or in an irregular non-repeating pattern. Particularly useful patterns are hexagonal patterns, rectilinear patterns, grid patterns, patterns having reflective symmetry, patterns having rotational symmetry, and the like. Asymmetric patterns can also be useful. The pitch can be the same between different pairs of nearest neighbor features or the pitch can vary between different pairs of nearest neighbor features. In particular examples, features of an array can each have an area that is larger than about 100 μm, about 250 μm, about 500 μm, about 1 μm, about 2.5 μm, about 5 μm, about 10 μm, about 100 μm, or about 500 μm. Alternatively, or additionally, features of an array can each have an area that is smaller than about 1 μm, about 500 μm, about 100 μm, about 25 μm, about 10 μm, about 5 μm, about 1 μm, about 500 μm, or about 100 μm. Indeed, a region can have a size that is in a range between an upper and lower limit selected from those exemplified above.

For examples that include an array of features on a surface, the features can be discrete, being separated by interstitial regions. The size of the features and/or spacing between the regions can vary such that arrays can be high density, medium density, or lower density. High density arrays are characterized as having regions separated by less than about 15 μm. Medium density arrays have regions separated by about 15 to about 30 μm, while low density arrays have regions separated by greater than about 30 μm. An array useful in one or more examples can have regions that are separated by less than about 100 μm, about 50 μm, about 10 μm, about 5 μm, about 1 μm or about 0.5 μm.

Target nucleic acids used in a method or composition of the present disclosure can be composed of DNA, RNA, or analogs thereof. The source of the target nucleic acids can be genomic DNA, messenger RNA, or other nucleic acids from native sources. In some cases, the target nucleic acids that are derived from such sources can be amplified prior to use in a method or composition herein. Target nucleic acids can optionally be derived from synthetic libraries. Synthetic nucleic acids can have native DNA or RNA compositions or can be analogs thereof.

Example biological samples from which target nucleic acids can be derived include, for example, those from a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium Falciparum. Target Nucleic Acids can Also be Derived from a Prokaryote such as a bacterium, Escherichia coli, staphylococci or Mycoplasma pneumoniae; an archae; a virus such as Hepatitis C virus or human immunodeficiency virus; or a viroid. Target nucleic acids can be derived from a homogeneous culture or population of the above organisms or alternatively from a collection of several different organisms, for example, in a community or ecosystem.

Target nucleic acids need not be derived from natural sources and can instead be synthesized using known techniques. For example, gene expression probes or genotyping probes can be synthesized and used to create an array in the methods set forth herein. In some examples, target nucleic acids can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid. For example, PCR amplification produces fragments having a size defined by the length of the fragment between the flanking primers used for amplification.

A population of target nucleic acids, or amplicons thereof, can have an average strand length that is desired or appropriate for a particular application of the methods or compositions set forth herein. For example, the average strand length can be less than about 100,000 nucleotides, about 50,000 nucleotides, about 10,000 nucleotides, about 5,000 nucleotides, about 1,000 nucleotides, about 500 nucleotides, about 100 nucleotides, or about 50 nucleotides. Alternatively, or additionally, the average strand length can be greater than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. The average strand length for population of target nucleic acids, or amplicons thereof, can be in a range between a maximum and minimum value set forth above. It will be understood that amplicons generated on a bead substrate (or otherwise made or used herein) can have an average strand length that is in a range between an upper and lower limit selected from those exemplified above.

In some cases, a population of target nucleic acids can be produced under conditions or otherwise configured to have a maximum length for its members. For example, the maximum length for the members that are used in a method set forth herein or that are present in a particular composition can be less than about 100,000 nucleotides, about 50,000 nucleotides, about 10,000 nucleotides, about 5,000 nucleotides, about 1,000 nucleotides, about 500 nucleotides, about 100 nucleotides or about 50 nucleotides. Alternatively, or additionally, a population of target nucleic acids, or amplicons thereof, can be produced under conditions or otherwise configured to have a minimum length for its members. For example, the minimum length for the members that are used in a method set forth herein or that are present in a particular composition can be more than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. The maximum and minimum strand length for target nucleic acids in a population can be in a range between a maximum and minimum value set forth above. It will be understood that amplicons generated on a bead surface can have maximum and/or minimum strand lengths in a range between the upper and lower limits exemplified above.

In particular examples, the target nucleic acids are sized relative to the area of the beads surface, for example, to facilitate kinetic exclusion. For example, the area for each of the sites of an array can be greater than the diameter of the excluded volume of the target nucleic acids in order to achieve kinetic exclusion. Taking, for example, examples that utilize an array of features on a surface, the area for each of the features can be greater than the diameter of the excluded volume of the target nucleic acids that are transported to the amplification sites. The excluded volume for a target nucleic acid and its diameter can be determined, for example, from the length of the target nucleic acid. Methods for determining the excluded volume of nucleic acids and the diameter of the excluded volume are described, for example, in U.S. Pat. No. 7,785,790; Rybenkov et al., Proc. Natl. Acad. Sci. U.S.A. 90: 5307-5311 (1993); Zimmerman et al., J. Mol. Biol. 222:599-620 (1991); or Sobel et al., Biopolymers 31:1559-1564 (1991), each of which is incorporated herein by reference in its entirety.

The following examples are intended to illustrate but not limit the present inventive subject matter.

### Example I

### High Specificity of Flow Cell Loading Via Streptavidin-Biotin Interaction or Hybridization

The example and accompanying image data of FIG. 2 of individual sequencing runs for (1) sample beads in suspension for flow cell loading via streptavidin-biotin interactions; (2) sample beads in suspension for flow cell loading via direct hybridization; and (3) sample beads in suspension for flow cell loading via indirect (or bridged) hybridization.

For each of the hybridization sample runs, the bead suspension was loaded manually in a saline sodium citrate buffer and Tween 20 surfactant. The buffer was tuned to a relatively high salt concentration to establish hybridization conditions. Each suspension was left on respective flow cells for 5-15 minutes at a common, static temperature between 20° C.-60° C. Each flow cell was then rinsed with buffer to remove any unbound beads. For each run sample beads were clustered with single strand DNA ligated with a P7 complement (cP7) primer adaptor on the 3′ end of the strand. For the direct hybridization sample, the flow cell was functionalized with P7 oligos for hybridization to the cP7 of the clustered DNA templates. For the indirect hybridization sample, the flow cell was functionalized with surface oligos having the same cP7 adapter sequence as the clustered DNA templates and then seeded with bridge oligos, each having two P7 adapters separated by a spacer sequence of desired length. The bridge oligos hybridize, at one end, to the surface oligos of the flow cell surface via P7/cP7 complementarity hybridize, at the opposite end to clustered DNA templates via P7/cP7 complementarity. In this manner, the bridge oligos mediate binding of the clustered beads to the surface oligos of the flow cell substrate.

For the streptavidin/biotin sample, beads in suspension were loaded in the same manner as described in the direct hybridization case, with the exception of the high salt conditions, which are unnecessary to streptavidin-biotin interactions. For the assay, a flow cell was coated with a monolayer of biotin groups clicked onto the hydrogel layer of the flow cell substrate and a layer of streptavidin was then added to the biotin layer via one of four available binding domains on the molecule, leaving the remaining three domains available to bind biotin moiety ligated to the DNA targets of the sample.

The image data of FIG. 2 demonstrates efficient flow cell loading of clustered beads both through high affinity Streptavidin-Biotin complex formation and direct and indirect hybridization interactions. Moreover, while the hybridization-based interactions capture beads at low concentrations, the hybridized sequences immobilizing the clustered beads are susceptible to desorption from the flow cell under low salt/high temperature conditions encountered during sequencing, and nonspecific binding (NSB) with non-complementary sequences. By contrast the Streptavidin-Biotin complex is more resilient and better suited to withstand sequencing reagents and conditions.

### Example II

### Selective Enrichment of Biotin-Incorporated Clustered Beads

The example and accompanying image and graphical data of FIG. 4 illustrate an example biotinylation method of the present disclosure and, further, demonstrate that biotin introduced during the clustering process yields clustered beads adapted for high stringency and high affinity capture by streptavidin-coated flow cells, with no evidence that biotinylation negatively affects qPCR yields. For the assay, a pool of AMO beads was coated and grafted with P7 primer and split into three batches. One batch was incubated with a red fluorescent cP7 to serve as an unclustered control. The other batch was split into two and clustered using non-bridging clustering-one batch with a biotinylated P5 primer in solution and the other with non-biotinylated P5 primer. The sub-batches were kept separate, and both incubated with a green fluorescent cP7 primer. After incubation, Beads were loaded using cBot for 10 minutes and then rinsed. Next, beads were loaded as follows—40 million beads per lane: grafted P7 only, clustered with 0%, 10%, 20%, or 30% biotinylated primer to total in-solution primer (lanes 4-8), or as a mixture—40 million P7 beads plus 20 million beads of one of the following: clustered with 0% biotin, 10% biotin, or 30% biotin (lanes 1-3). The flow cell was imaged by light microscopy in both the red (P7 only) and green (clustered) channels. Light micrographs of the 8 flow cell lanes show excellent enrichment of clustered, biotinylated beads (FIG. 4a, “M” signifies “million” beads). Lanes 8 and 7 of the flow cell show that control P7 beads or clustered beads with no biotin do not bind non-specifically to the flow cell. Lanes 4-6 show that biotin-incorporated clustered beads bind to the flow cell, with 20% biotinylated primer performing better than 10%, and about the same as 30% (percent signifies the percentage of in-solution P5 primer containing biotin in comparison with the total in-solution primer quantity). When P7 control beads are combined with clustered beads in a 2:1 ratio before flow cell capture, there is almost no signal coming from the P7 beads, and at 30% biotin-P5 loading, the number of beads per frame is the expected amount (half of the signal in lane 4, since half the clustered beads were loaded). The bead count quantification is more clearly represented in FIG. 4b, which also demonstrates the beads are not desorbed by LDR (formamide) treatment at 60° C. for ˜4 minutes. To ensure biotin incorporation was not negatively affecting the clustering process, qPCR was performed on clustered beads incorporating 10%, 20%, 30%, or 0% biotin (FIG. 4c). No significant differences were seen in any conditions tested, indicating that biotin does not affect the clustering process.

### Example III

### Hybridization/Extension Assay

Hybridization assays were performed, separately, on biotinylated DNA sample (10 bp) targets and non-biotinylated analogs to quantify relative hybridization during qPCR amplification. Clones were amplified from a qPCR primer manufactured by IDT that included a black hole quencher (BHQ) annealed to a fluorescent (FAM) P5 template at a ratio of 1.1:1 BHQ:FAM.

With reference to FIG. 5(a), within each reaction well of a 96-well half-area plate, 20 uL of “Reaction Buffer” (16.7 uL water, 2 uL Tris buffer pH 8.6 (1 M) was mixed with 0.1 uL duplex pol substrate (10 mM), 0.6 uL dNTPs (100 mM), and 0.61 uL Bsu polymerase (165 U/uL)) with 5 uL of 1 μM P5 primer plus 15 uL of water. The plate was positioned on a plate reader and pre-warmed to 38° C. for 5-10 minutes. The reaction was then initiated on the plate reader by injecting 10 uL of 40 mM MgOAc into each well (bringing the total volume to 50 uL per reaction well) while maintaining a temperature of 38° C. Fluorescence at 490 nm excitation and 525 nm emission was read every 39 seconds over the course of 1 h. The resulting scatter plot of fluorescence data of FIG. 5(b) shows there is no appreciable difference in relative hybridization over time between biotinylated amplicons and non-biotinylated analogs, meaning that hybridization-extension is not significantly impacted by incorporation of Biotin, Q.E.D.

1. A method for performing analysis on target nucleic acids, the method comprising:
clustering, in a solution or suspension, a clonal amplicon population from each of a plurality of nucleic acid targets seeded on an array of bead substrates,
wherein a desired portion of each clonal amplicon population is conjugated with a capture agent, and wherein clustering results in a pool of clustered bead substrates;
providing a flow cell substrate functionalized with a coating of binding partners on at least a portion of the substrate,
wherein the binding partner has a high affinity for binding the capture agent;
loading the pool of clustered bead substrates onto the flow cell;
immobilizing at least a portion of the bead substrates onto the flow cell substrate via interactions between the binding agents of the clustered beads substrates and binding partners of the functionalized substrate of the flow cell; and
performing sequence analysis on the immobilized bead substrates.

2. The method of claim 1 further comprising introducing a cleaving reagent to remove the immobilized substrates at the end of an analysis cycle,
wherein the cleaving reagent removes the immobilized substrates under conditions that substantially conserve functionality of the coating of binding partners on the flow cell surface for multi-cycle use.

3. The method of claim 1, wherein the capture agent comprises a biotin moiety.

4. The method of claim 3, wherein the binding partner comprises a streptavidin moiety.

5. The method of claim 4 wherein the interactions between the biotin moiety and streptavidin moiety comprise formation of an orthogonal complex and result in the immobilization of at least a portion of the bead substrates onto the flow cell substrate.

6. The method of claim 4 wherein the flow cell substrate is functionalized with a coating of biotin moieties interposed between the flow cell substrate and the coating of streptavidin moieties,
wherein each streptavidin moiety comprises four binding domains each characterized by high binding affinity for biotin,
wherein a plurality of biotin moieties of the coating of biotin moieties each interact with a respective binding domain of a corresponding plurality of streptavidin moieties of the coating of streptavidin moieties interact to form a corresponding plurality of orthogonal complexes, and
wherein at least each of the corresponding plurality of streptavidin moieties are configured to immobilize a clustered bead onto the flow cell substrate via interactions between one or more biotin moieties of the clustered bead and one or more of the three remaining binding domains of each streptavidin moiety.

7. The method of claim 1, wherein the amplicons of the clonal amplicon population comprise double-stranded DNA molecules.

8. The method of claim 7, wherein each of a plurality of the double-stranded DNA molecules is cross-linked with one or more photoactive moieties.

9. The method of claim 8, wherein the one or more photoactive moieties comprise a vinylcarbazole group.

10. A method for performing analysis on target nucleic acids, the method comprising:
mixing a first reagent mixture with an amount of double stranded target nucleic acids within a reservoir to define a first solution;
wherein the 5′ and 3′ ends of target nucleic acids are ligated with respective first and second adapters,
and wherein the first reagent mixture comprises nucleoside triphosphates (NTPs), one or more replication enzymes, and adapter primers, wherein the adaptor primers include a primer mix of second adaptor primers and analogs primers conjugated with a capture agent in a desired ratio;
mixing the first solution with a second solution (or suspension) comprising primer grafted beads,
wherein each primer is configured to hybridize to the first adapter of the target nucleic acids;
clustering to produce a pool of clustered beads,
wherein target nucleic acids are seeded onto primer grafted beads and amplified to form clonal populations,
wherein the rate of amplification rate preferably exceeds the rate at which target nucleic acids are seeded,
wherein each clonal population comprises amplicons synthesized with either a second adaptor primer or the primer analog conjugated with a capture agent, wherein the proportion of amplicons synthesized with the capture agent conjugate corresponds to the desired ratio of the primer mix,
and wherein clustering yields a bead pool comprising well-clustered beads characterized by high occupancy of amplicons synthesized with the capture agent conjugate, poorly clustered beads characterized by low occupancy of amplicons synthesized with the capture agent conjugate relative to well-clustered beads, and unclustered beads lacking amplicons synthesized with the capture agent conjugate;
loading the bead pool onto a flow cell substrate, at least a portion of which is functionalized with a coating of binding partners that bind capture agents with high affinity such that well-clustered beads of the bead pool are selectively captured on the functionalized surface of the flow cell, thereby enabling sorting of well-clustered beads from poorly clustered and unclustered beads from the bead pool; and
enriching the bead pool by removing poorly clustered and unclustered beads from the functionalized substrate of the flow cell.

11. The method of claim 10 further comprising introducing a cleaving reagent to remove the immobilized substrates at the end of an analysis cycle,
wherein the cleaving reagent removes the immobilized substrates under conditions that substantially conserve functionality of the coating of binding partners on the flow cell surface for multi-cycle use.

12. The method of claim 10, wherein the capture agent comprises a biotin moiety.

13. The method of claim 12, wherein the binding partner comprises a streptavidin moiety.

14. The method of claim 13 wherein the interactions between the biotin moiety and streptavidin moiety comprise formation of an orthogonal complex and result in the immobilization of at least a portion of the bead substrates onto the flow cell substrate.

15. The method of claim 13 wherein the flow cell substrate is functionalized with a coating of biotin moieties interposed between the flow cell substrate and the coating of streptavidin moieties,
wherein each streptavidin moiety comprises four binding domains each characterized by high binding affinity for biotin,
wherein a plurality of biotin moieties of the coating of biotin moieties each interact with a respective binding domain of a corresponding plurality of streptavidin moieties of the coating of streptavidin moieties interact to form a corresponding plurality of orthogonal complexes, and
wherein at least each of the corresponding plurality of streptavidin moieties are configured to immobilize a clustered bead onto the flow cell substrate via interactions between one or more biotin moieties of the clustered bead and one or more of the three remaining binding domains of each streptavidin moiety.

16. The method of claim 10, wherein the amplicons of the clonal amplicon population comprise double-stranded DNA molecules.

17. The method of claim 16, wherein each of a plurality of the double-stranded DNA molecules is cross-linked with one or more photoactive moieties.

18. The method of claim 17, wherein the one or more photoactive moieties comprise a vinylcarbazole group.
